SEQUENCE LISTING (26 sequences)

SEQ ID NO: 1 - DNA, 18 bp, Unknown
  Primer
ggtctttgacaacaagca

SEQ ID NO: 2 - DNA, 17 bp, Unknown
  Primer
tgatggttctggcgata

SEQ ID NO: 3 - DNA, 21 bp, Unknown
  Primer
cgggagacaagaactacatca

SEQ ID NO: 4 - DNA, 19 bp, Unknown
  Primer
cgtagagggcactggagaa

SEQ ID NO: 5 - DNA, 18 bp, Unknown
  Primer
cccatcaaaaccaagcaa

SEQ ID NO: 6 - DNA, 20 bp, Unknown
  Primer
tcaacactcatctccacgaa

SEQ ID NO: 7 - DNA, 20 bp, Unknown
  Primer
gaccataggcttcgctgtct

SEQ ID NO: 8 - DNA, 20 bp, Unknown
  Primer
aggttgatgaactgctcgga

SEQ ID NO: 9 - DNA, 22 bp, Unknown
  Primer
cgactggagacactgttcagga

SEQ ID NO: 10 - DNA, 24 bp, Unknown
  Primer
cttcaggcagattggttatcactt

SEQ ID NO: 11 - DNA, 21 bp, Unknown
  Primer
gctgtcgtctctgaagtggtt

SEQ ID NO: 12 - DNA, 23 bp, Unknown
  Primer
ggcatcgtcaagttctttgttgt

SEQ ID NO: 13 - DNA, 20 bp, Unknown
  Primer
ccaccacttcggctttgaag

SEQ ID NO: 14 - DNA, 26 bp, Unknown
  Primer
ggaaaagtcagactacgtctacgaaa

SEQ ID NO: 15 - DNA, 18 bp, Unknown
  Primer
tgcagccgccatttcttc

SEQ ID NO: 16 - DNA, 20 bp, Unknown
  Primer
tagcgctgttggacgctcta

SEQ ID NO: 17 - DNA, 20 bp, Unknown
  Primer
tcaacactcatctccacgaa

SEQ ID NO: 18 - DNA, 18 bp, Unknown
  Primer
caccacagcaaccacatc

SEQ ID NO: 19 - DNA, 19 bp, Unknown
  Primer
cagaaatgggaacagacaa

SEQ ID NO: 20 - DNA, 21 bp, Unknown
  Primer
gacttcataggcactctcaac

SEQ ID NO: 21 - DNA, 20 bp, Unknown
  Primer
tgaggaagcggaggaacaat

SEQ ID NO: 22 - DNA, 18 bp, Unknown
  Primer
tgggactcggcaatgtca

SEQ ID NO: 23 - DNA, 20 bp, Unknown
  Primer
caatgacctgacaacccaga

SEQ ID NO: 24 - DNA, 22 bp, Unknown
  Primer
ccttgacaactgagacaccaga

SEQ ID NO: 25 - DNA, 17 bp, Unknown
  Primer
accagccgcaacgtgaa

SEQ ID NO: 26 - DNA, 18 bp, Unknown
  Primer
ccttcgcgcctctgatca

(19) United States
(12) Patent Application Publication
(10) Pub. No.: US 2015/0344974 A1
(43) Pub. Date: Dec. 3, 2015

(54) DNA MARKERS FOR FEED EFFICIENCY IN CATTLE

(71) Applicant: The Texas A&M University System, College Station, TX (US)

(72) Inventors: PENNY K. RIGGS, Bryan, TX (US); Robert N. Vaughn, College Station, TX (US)

(21) Appl. No.: 14/724,696

(22) Filed: May 28, 2015

Related U.S. Application Data
(60) Provisional application No. 62/006,740, filed on Jun. 2, 2014.

(51) Int. Cl. C12Q 1/68

(52) U.S. Cl. CPC C12Q 1/6888; C12Q 2600/172; C12Q 2600/156; C12Q 2600/124

(57) ABSTRACT

The present invention provides genetic polymorphisms and methods for identifying said genetic polymorphisms associated with feed efficiency in cattle, as well as kits for identifying said polymorphisms in beef cattle. The invention also provides methods of predicting the feed efficiency of beef in a head of cattle based on the presence of a hapblock conferring feed efficiency. In other embodiments, the invention provides methods of determining a breeding value for a head of cattle involving detection of such polymorphisms.

### CROSS REFERENCE TO RELATED APPLICATIONS

This application claims priority to U.S. Provisional Application No. 62/006,740, filed Jun. 2, 2014, which is herein incorporated by reference in its entirety.

### STATEMENT REGARDING FEDERALLY FUNDED RESEARCH

This invention was made with government support under grant no. 2008-35205-18767 awarded by USDA National Institute of Food and Agriculture Animal Genome Program. The government has certain rights in the invention.

### FIELD OF THE INVENTION

The present invention relates generally to the field of mammalian genetics. More particularly, the invention concerns genetic markers for the selection of cattle having a genetic predisposition for progeny with improved feed efficiency traits.

### INCORPORATION OF SEQUENCE LISTING

The sequence listing that is contained in the file named “TAMC031US_ST25,” which is 5 kilobytes as measured in Microsoft Windows operating system and was created on May 26, 2015, is filed electronically herewith and incorporated herein by reference.

### BACKGROUND OF THE INVENTION

Efficient use of resources continues to be regarded as a critical area of emphasis for livestock production. Feed costs can contribute as much as 70% of total livestock production costs. Environmental costs associated with beef production have become increasingly important to the consumer. Although the beef industry has made substantial strides in reducing the environmental footprint of cattle production, feed efficiency remains a major concern for beef producers.

### SUMMARY OF THE INVENTION

In one aspect, the invention provides a method of selecting a head of beef cattle with a genetic predisposition for increased feed efficiency comprising selecting said head of beef cattle based on the presence in the genome of at least one genetic haplotype conferring said increased feed efficiency selected from the group consisting of (a) a haplotype located in a region on bovine chromosome 29 defined by positions 45230630 and 45242406; (b) a haplotype located in a region on bovine chromosome 2 defined by positions 103881402 and 103950562; and (c) a haplotype located in a region on bovine chromosome 16 defined by positions 27781671 and 27840011. In certain embodiments, said haplotype of (a) comprises the ACTN3 gene, or said haplotype of (b) comprises the FN1 gene, or said haplotype of (c) comprises the CAPN2 gene. In another embodiment, such a method comprises selecting said head of beef cattle based on the presence in the genome of at least two of said haplotypes. In other embodiments, said haplotype of (a) is inherited from the maternal or the paternal parent, or is inherited from the genome of a Nellore head of cattle. In other embodiments, said haplotype of (b) is inherited from the paternal parent, or is inherited from the genome of an Angus head of cattle. In still further embodiments, said haplotype of (c) is inherited from the maternal parent, or is inherited from the genome of a Nellore head of cattle. In another embodiment, such a method further comprises sequencing said haplotype. In other embodiments, the method further comprises detecting at least one SNP within or genetically linked to said at least one genetic haplotype, wherein said SNP is set forth in Tables 6-8, or further comprises detecting all SNPs within or genetically linked to said haplotype.

In still other embodiments, the method further comprises genotyping at least one parent of said head of beef cattle for the presence of said at least one genetic haplotype, or genotyping both parents of said head of beef cattle. In other embodiments, said head of beef cattle is a Bos indicus or a Bos taurus head of beef cattle, or is a hybrid of a Bos indicus species and a Bos taurus species. In another embodiment, said Bos indicus species further comprises a Nellore head of cattle, or said Bos taurus species further comprises an Angus head of cattle. In another embodiment, the method further comprises genotyping a population of beef cattle for the presence of said at least one genetic haplotype. In other embodiments, the method further comprises breeding the selected head of beef cattle comprising said at least one genetic haplotype with a second head of beef cattle to obtain a progeny head of beef cattle with increased feed efficiency relative to a head of beef cattle of the same breed lacking said at least one genetic haplotype, or breeding the progeny head of beef cattle with increased feed efficiency to a second head of beef cattle to produce a progeny of a further generation comprising said increased feed efficiency. In other embodiments, the presence of said at least one genetic haplotype conferring said increased feed efficiency is detected by assaying of genetic material from the head of beef cattle, or said assaying is carried out by PCR.

In another aspect, the invention provides a method of predicting increased feed efficiency in a head of beef cattle comprising genotyping the head of beef cattle for the presence in the genome of at least one genetic haplotype conferring said increased feed efficiency selected from the group consisting of (a) a haplotype located in a region on bovine chromosome 29 defined by positions 45230630 and 45242406; (b) a haplotype located in a region on bovine chromosome 2 defined by positions 103881402 and 103950562; and (c) a haplotype located in a region on bovine chromosome 16 defined by positions 27781671 and 27840011. In some embodiments, said head of beef cattle is a Bos indicus or a Bos taurus head of beef cattle, or said head of beef cattle is a hybrid between a Bos indicus species and a Bos taurus species, or said Bos indicus species further comprises a Nellore head of cattle, or said Bos taurus species further comprises an Angus head of cattle.

In another aspect, the invention provides a method of determining the breeding value of a head of cattle comprising: (a) genotyping the head of beef cattle to determine the presence in the genome of at least one genetic haplotype conferring said increased feed efficiency selected from the group consisting of (i) a haplotype located in a region on bovine chromosome 29 defined by positions 45230630 and 45242406; (ii) a haplotype located in a region on bovine chromosome 2 defined by positions 103881402 and 103950562; and (iii) a haplotype located in a region on bovine chromosome 16 defined by positions 27781671 and 27840011; (b) determining an estimated breeding value for the individual; and (c) selecting at least a first individual with a desired estimated breeding value for breeding. In one embodiment, the method further comprises (d) breeding said individual to a second individual to obtain progeny.

In another aspect, the invention provides a kit for identifying a head of beef cattle with increased feed efficiency, the kit comprising: (a) at least one primer that amplifies a SNP within or genetically linked to at least one haplotype conferring said increased feed efficiency selected from the group consisting of (i) a haplotype located in a region on bovine chromosome 29 defined by positions 45230630 and 45242406; (ii) a haplotype located in a region on bovine chromosome 2 defined by positions 103881402 and 103950562; and (ii) a haplotype located in a region on bovine chromosome 16 defined by positions 27781671 and 27840011; wherein said SNP is set forth in Tables 6-8; and (b) a reaction reagent.

### BRIEF DESCRIPTION OF THE DRAWINGS

FIG. 1—Shows a graph of qRT-PCR results demonstrating mRNA expression of ACTN3 in cattle with high and low efficiency. Expression of ACTN3 was 1.6-fold greater in the low efficiency group.

FIG. 2—Shows a correlation between proportion of fast to slow twitch (Type IIx/Type I) fiber ratio and expression of actinin 3 (r=0.622, P<0.001).

### BRIEF DESCRIPTION OF THE SEQUENCES

SEQ ID NOs:1-26—Sequences of primers used for qRT-PCR.

### DETAILED DESCRIPTION OF THE INVENTION

The invention provides, in one aspect, methods and compositions for the improvement of beef cattle with respect to feed efficiency. The invention provides for the first time a genetic haplotype harboring detectable single nucleotide polymorphisms (SNPs) and that confers increased feed efficiency in beef cattle. Thus, in one embodiment, the invention provides a method of selecting a head of beef cattle with increased feed efficiency comprising selecting a head of beef cattle based on the presence in the genome of a genetic marker indicating the presence of a genetic haplotype conferring increased feed efficiency.

The present invention represents a significant advance by providing genetic markers and associated methods for improvement in cattle production efficiency with simultaneous reduction in emissions. The environmental costs associated with beef production have become an increasingly important concern for the consumer. A reduction in feed input is one possible method to reduce emissions caused by grain production and reduce the overall environmental impact of beef production.

As described in the Examples below, skeletal muscle gene expression networks were examined in F2 Nellore-Angus steers that were evaluated for feed efficiency in order to identify genetic variation in expression of genes related to muscle physiology and homeostasis that are important for overall feed efficiency. Feed efficiency applies to some measure of output to input and can be measured in a variety of ways. For example, gain to feed ratio (G:F) measures the amount of total gain by weight of the animal compared to feed required to achieve that gain, and average daily gain (ADG) is a measure of total weight gain over a set period of time. Residual feed intake (RFI) models daily intake based on ADG and metabolic mid-weight.

As described herein, a genetic locus (“haplotype block” or “hapblock”) was identified that contributes significantly to overall feed efficiency and contains SNPs that can be used as genetic markers for the feed efficiency trait. Furthermore, the applicants found that the hapblock described herein is heritable and thus can be successfully introduced into any desired cattle background or breed. Thus, in one embodiment, the methods of the invention provide for identification of new loci that can be tracked with genetic markers in beef cattle to yield a head of cattle with increased feed efficiency relative to cattle lacking the relevant haplotype. Such a method may serve as a genetic test for predicting the feed efficiency of a head of cattle.

A haplotype in accordance with the invention may be selected from the group consisting of (a) a haplotype located in a region on bovine chromosome 29 defined by genome positions 45230630 and 45242406; (b) a haplotype located in a region on bovine chromosome 2 defined by positions 103881402 and 103950562; and (c) a haplotype located in a region on bovine chromosome 16 defined by positions 27781671 and 27840011, including any combinations thereof. In another embodiment, a head of cattle may comprise more than one haplotype conferring increased feed efficiency. Also provided are methods of determining a breeding value for a head of beef cattle comprising (a) genotyping the head of beef cattle to determine the presence in the genome of at least one genetic haplotype conferring said increased feed efficiency as set forth herein; (b) determining an estimated breeding value for the individual; and (c) selecting at least a first individual with a desired estimated breeding value for breeding.

In accordance with the invention, a haplotype conferring increased feed efficiency in beef cattle may be inherited from either the maternal parent or the paternal parent of a Bos indicus or Bos taurus breed of cattle. In certain embodiments, inheritance of a haplotype described herein from a particular parent may confer increased feed efficiency relative to inheritance from the other parent. For example, in one embodiment, inheritance of a haplotype located in a region on bovine chromosome 29 defined by genome positions 45230630 and 45242406 may confer increased feed efficiency when inherited from the maternal parent. In another embodiment, inheritance of such a haplotype may confer increased feed efficiency when inherited from the paternal parent. In another embodiment, inheritance of a haplotype located in a region on bovine chromosome 2 defined by positions 103881402 and 103950562 may confer increased feed efficiency when inherited from the paternal parent. In another embodiment, inheritance of a haplotype located in a region on bovine chromosome 16 defined by positions 27781671 and 27840011 may confer increased feed efficiency when inherited from the maternal parent. Inheritance from either parent, however, may be used in accordance with the invention to confer an improved phenotype.

In other embodiments, inheritance of a haplotype in accordance with the invention from a specific lineage or genome may confer increased feed efficiency relative to inheritance from another lineage or genome. For example, in one embodiment, inheritance of a haplotype located in a region on bovine chromosome 29 defined by positions 45230630 and 45242406 may confer increased feed efficiency when the haplotype originates from a B. indicus lineage or genome. In another embodiment, inheritance of such a haplotype may confer increased feed efficiency when the haplotype originates from a B. taurus lineage or genome. In specific embodiments, such a haplotype may be inherited from a Nellore breed of cattle or from an Angus breed of cattle. In another embodiment, inheritance of a haplotype located in a region on bovine chromosome 2 defined by positions 103881402 and 103950562 may confer increased feed efficiency when the haplotype originates from a B. taurus lineage or genome. In a specific embodiment, such a haplotype may be inherited from an Angus breed of cattle. In another embodiment, inheritance of a haplotype located in a region on bovine chromosome 16 defined by positions 27781671 and 27840011 may confer increased feed efficiency when the haplotype originates from a B. indicus lineage or genome. In a specific embodiment, such a haplotype may be inherited from a Nellore breed of cattle. Additional haplotypes conferring increased feed efficiency in accordance with the invention are also within the scope of the invention.

In further embodiments, a haplotype as described herein may comprise or be genetically linked to certain genes or markers useful in accordance with the present invention. For example, in an embodiment, a haplotype located in a region on bovine chromosome 29 defined by positions 45230630 and 45242406 conferring increased feed efficiency may comprise an actinin gene, such as a gene encoding actinin-3 (ACTN3). In another embodiment, a haplotype located in a region on bovine chromosome 2 defined by positions 103881402 and 103950562 conferring increased feed efficiency may comprise or be genetically linked to a fibronectin gene, such as a gene encoding fibronectin 1 (FN1). In another embodiment, a haplotype located in a region on bovine chromosome 16 defined by positions 27781671 and 27840011 conferring increased feed efficiency may comprise or be genetically linked to a calpain gene, such as a gene encoding calpain 2 (CAPN2).

In certain other embodiments, the invention provides a genetic locus or haplotype from a Bos indicus or Bos taurus beef cattle breed that confers increased feed efficiency. This haplotype can be introduced into cattle of different cattle breeds or species to produce cattle that comprise the trait of increased feed efficiency. The locus of interest can be present in any species or breed of cattle as set forth herein. One of skill in the art will understand that other breeds of beef cattle may be useful in accordance with the invention.

In other embodiments, a haplotype of interest in the present invention may comprise a polymorphism within or genetically linked to a haplotype set forth herein. For example, a polymorphism in accordance with the invention may comprise a polymorphism set forth herein as being located in or genetically linked to a region on bovine chromosome 29 defined by positions 45230630 and 45242406, or located in or genetically linked to a region on bovine chromosome 2 defined by positions 103881402 and 103950562, or located in or genetically linked to a region on bovine chromosome 16 defined by positions 27781671 and 27840011. For example, a polymorphism of the invention may comprise a SNP as set forth in Tables 6-8. In a specific embodiment, a haplotype or hapblock as described herein may comprise a plurality of polymorphisms or SNPs. Such SNPs may be inherited together to produce a head of cattle with increased feed efficiency. For example, a haplotype or hapblock may comprise a single SNP, or may comprise 2, 3, 4, 5, 6, 7, 8, 9, 10, 11, 12, 13, 14, 15, 16, 17, 18, 19, 20, or more SNPs that may be inherited together to produce a head of cattle with increased feed efficiency.

### I. Genetic Assays and Selections

The techniques of the invention are significant in that they allow improvement of beef cattle for a trait that is important to both consumers and producers of beef without the need for costly or unreliable phenotypic assays and manual breeding selections. With increasing costs associated with animal breeding and artificial insemination, each head of cattle produced represents a substantial investment of time and money. Traditional methods of breeding cattle can lack accuracy due to environmental variance or scoring error. Further, complex gene action and interactions among genes can complicate breeding. Phenotypic selection often does not efficiently take into account such genetic variability. Selection based on DNA tests is therefore significant in that it allows for selection of beef cattle with increased feed efficiency without the cost and lack of reliability of conventional assays or selections.

The use of genetic assays as described herein to identify a haplotype or polymorphism associated with increased feed efficiency will find use in breeding or selecting of beef cattle produced for slaughter, e.g., for production of meat products. Thus, one embodiment of the invention comprises a breeding program directed at enhancement of desirable characteristics in beef cattle breeds adapted for meat production, as opposed to cattle specifically suited or used for production of dairy products. Such techniques have to date been largely lacking for beef cattle.

Genetic assay-assisted selections for animal breeding are important in that they allow selections to be made without the need for raising and phenotypic testing of progeny. In particular, such tests allow selections to occur among related individuals that do not necessarily exhibit the trait in question and that can be used in introgression strategies to select both for the trait to be introgressed and against undesirable background traits. However, it has been difficult to identify genetic assays for loci yielding highly heritable traits of large effect, particularly as many such traits may not be segregating and may already be fixed with near optimal alleles in commercial lines. The invention overcomes this difficulty by providing such assays for alleles of hapblocks that are segregating in beef cattle populations.

In accordance with the invention, any assay which sorts and identifies animals based upon differences in hapblock alleles or SNPs within or genetically linked thereto may be used and is specifically included within the scope of this invention. One of skill in the art will recognize that, having identified a hapblock or associated polymorphism for a particular associated trait, there are an essentially infinite number of ways to genotype animals for this hapblock or polymorphism. These tests may be made at the nucleic acid and/or protein level. The design of such alternative tests merely represents a variation of the techniques provided herein and is thus within the scope of this invention as fully described herein. Illustrative procedures are described herein below, but one of skill in the art will recognize that other techniques or methods may also be used in accordance with the invention.

Samples obtained from an individual can be analyzed for the presence of hapblocks or SNPs associated with increased feed efficiency using any suitable method, including microarray. In addition, several methods and different genotyping platforms that are used for SNP genotyping are known in the art (e.g., TaqMan, Pyrosequencing, RFLP, Direct Sequencing, etc.). Microarrays or chips can contain thousands up to a million or a little over one million SNPs, which are also used for genotyping.

Alternatively, or in addition to, analyzing a sample for the presence of hapblocks or SNPs associated with increased feed efficiency, a sample may be analyzed for the presence of at least one gene in which a particular SNP resides, or a gene product encoded by a gene in which a particular SNP resides. Gene products as used herein refer to any molecule produced as a result of gene expression, and may include proteins, mRNAs, tRNAs, microRNAs, or the like. The at least one gene may include, but is not limited to ACTN3, FN1, and CAPN2. In some embodiments, the at least one gene may comprise more than one gene, or any number of genes identified as useful in accordance with the invention. In another embodiment, additional genes useful in accordance with the invention may be identified in a genetic pathway such as a skeletal muscle pathway or other pathway that confer or are genetically linked to a trait of interest. SNPs of interest may be identified within or generically linked to such genes for use with the invention. Methods of analyzing a sample for the presence of a haplotype and/or SNP and/or gene and/or gene product are well known in the art. As described herein, such methods may include PCR for verifying the presence of a particular haplotype and/or gene and/or SNP, direct sequencing, or real-time PCR for detecting the expression of a gene in different cells or tissues.

Non-limiting examples of methods for identifying the presence or absence of a hapblock or polymorphism include single-strand conformation polymorphism (SSCP) analysis, RFLP analysis, heteroduplex analysis, denaturing gradient gel electrophoresis, temperature gradient electrophoresis, ligase chain reaction, and direct sequencing of the gene. Techniques employing PCR detection are advantageous in that detection is more rapid, less labor intensive, and requires smaller sample sizes. The techniques and primers that may be used in this regard are well known in the art. A PCR-amplified portion of, for example, the ACTN3 gene, or the FN1 gene, or the CAPN2 gene, or a surrounding region, may be screened for a polymorphism, for example, with direct sequencing of the amplified region, by microarray, by detection of restriction fragment length polymorphisms produced by contacting the amplified fragment with a restriction endonuclease having a cut site altered by the polymorphism, by allele-specific PCR in which the alleles are individually amplified by specific oligonucleotide primers, by SSCP analysis of the amplified region, or by other methods known in the art. These techniques may also be carried out directly on genomic nucleic acids without the need for PCR amplification, although in some applications this may require more labor. In accordance with the invention, a region surrounding a gene of interest, such as ACTN3, FN1, or CAPN2, which may be useful for identification of SNPs linked to increased feed efficiency may be found within a distance of approximately 1 Mb of the gene of interest. For example, SNPs may be identified within about 1 kb, 2 kb, 3 kb, 4 kb, 5 kb, 10 kb, 15 kb, 20 kb, 25 kb, 50 kb, 75 kb, 100 kb, 200 kb, 250 kb, 500 kb, 750 kb, or 1 Mb or more from a gene of interest.

Once an assay format has been chosen, selections may be unambiguously made based on genotypes assayed at any time after a nucleic acid or protein sample can be collected from an individual, such as an adult, or a young animal, or even earlier in the case of testing of embryos in vitro, or testing of fetal offspring. Any source of genetic material (including, for example, DNA and RNA) or a product encoded or produced thereby may be analyzed for scoring of genotype. In one embodiment of the invention, nucleic acids are screened that have been isolated from the hair roots, blood, muscle tissue, or semen of the bovine analyzed. Peripheral white blood cells may be conveniently used as a source of genetic material, and the genetic material may be DNA. A sufficient amount of cells may be obtained to provide a sufficient amount of DNA for analysis, although only a minimal sample size will be needed where scoring is by amplification of nucleic acids. DNA may be isolated from blood cells or other cell or tissue types by standard nucleic acid isolation techniques known to those skilled in the art.

In genetic assay-assisted breeding, eggs may be collected from selected females and in vitro fertilized using semen from selected males and implanted into the selected female or into another female for gestation and birth. Assays may be advantageously used with both male and female cattle. Using in vitro fertilization, genetic assays may be conducted on developing embryos at the 4-8 cell stage, for example, using PCR, and selections made accordingly. Embryos can thus be selected that are homozygous or heterozygous for the desired haplotype or marker prior to embryo transfer.

Alternatively, genetic assay-assisted breeding may comprise artificial insemination methods. In particular, semen from a selected male may be artificially introduced to the reproductive tract of a selected female to result in in vivo fertilization. Use of genotype-assisted selection provides more efficient and accurate results than traditional methods. This also allows rapid introduction into or elimination from a particular genetic background of the specific trait or traits associated with the identified genetic marker. In the present case, screening for haplotype alleles conferring increased feed efficiency may be used to allow the efficient culling of cattle without increased feed efficiency, and the selection of cattle with increased feed efficiency, as desired.

Genetic assays can be used to obtain information about the genes that influence an important trait, thus facilitating breeding efforts. Factors considered in developing markers for a particular trait include: how many genes influence a trait, where the genes are located on the chromosomes (e.g., near which genetic markers), how much each locus affects the trait, whether the number of copies has an effect (gene dosage), pleiotropy, environmental sensitivity, and epistasis.

A genetic map represents the relative order of genetic markers, and their relative distances from one another, along each chromosome of an organism. During sexual reproduction in higher organisms, the two copies of each chromosome pair align themselves closely with one another. Genetic markers that lie close to one another on the chromosome are seldom recombined, and thus are usually found together in the same progeny individuals. Markers that lie close together show a small percent recombination, and are said to be linked. Markers linked to loci having phenotypic effects are particularly important in that they may be used for selection of individuals having the desired trait.

The identity of a given allele can therefore be determined by identifying nearby genetic markers that are usually co-transmitted with the gene from parent to progeny. This principle applies both to genes with large effects on phenotype (simply inherited traits) and genes with small effects on phenotype. As such, by identifying a marker linked to a particular trait, this will allow direct selection for the linked polymorphism without the need for detecting that particular polymorphism due to genetic linkage between the traits. Those of skill in the art will therefore understand that when genetic assays for a haplotype or marker set forth herein are mentioned, this specifically encompasses detection of genetically linked polymorphisms that are informative for the haplotype or marker allele. Such polymorphisms have predictive power relative to the trait to the extent that they also are linked to the contributing locus for the trait. Such markers thus also have predictive potential for the trait of interest. In this regard, a polymorphism of interest in the present invention may comprise a polymorphism in a haplotype located in a region on bovine chromosome 29 defined by positions 45230630 and 45242406, or located in a region on bovine chromosome 2 defined by positions 103881402 and 103950562, or located in a region on bovine chromosome 16 defined by positions 27781671 and 27840011, or a SNP located within or genetically linked to this haplotype. For example, a polymorphism in accordance with the invention may comprise a polymorphism set forth in Tables 6-8.

Most natural populations of animals are genetically quite different from the classical linkage mapping populations. While linkage mapping populations are commonly derived from two-generation crosses between two parents, many natural populations are derived from multi-generation matings between an assortment of different parents, resulting in a massive reshuffling of genes. Individuals in such populations carry a complex mosaic of genes, derived from a number of different founders of the population. Gene frequencies in the population as a whole may be modified by natural or artificial selection, or by genetic drift (e.g., chance) in small populations. Given such a complex population with superior average expression of a trait, a breeder might wish to (1) maintain or improve the expression of the trait of interest, while maintaining desirable levels of other traits; and (2) maintain sufficient genetic diversity that rare desirable alleles influencing the trait(s) of interest are not lost before their frequency can be increased by selection.

Genetic assays may find particular utility in maintaining sufficient genetic diversity in a population while maintaining favorable alleles. For example, one might select a fraction of the population based on favorable phenotype (perhaps for several traits—one might readily employ index selection), then apply genetic assays as described herein to this fraction and keep a subset that represents much of the allelic diversity within the population. Strategies for extracting a maximum of desirable phenotypic variation from complex populations remain an important area of breeding strategy. An integrated approach, merging classical phenotypic selection with a genetic marker-based analysis, may aid in extracting valuable genes from heterogeneous populations.

The techniques of the present invention may potentially be used with any bovine, including Bos taurus and Bos indicus cattle. In particular embodiments of the invention, the techniques described herein are specifically applied for selection of beef cattle, as the genetic assays described herein will find utility in maximizing production of animal products, such as meat. As used herein, the term “beef cattle” refers to cattle grown or bred for production of meat or other non-dairy animal products. Therefore, a “head of beef cattle” refers to at least a first bovine animal grown or bred for production of meat or other non-dairy animal products. Examples of breeds of cattle that may be used with the invention include, but are not limited to, Africander, Alberes, Alentejana, American, American White Park, Amerifax, Amrit Mahal, Anatolian Black, Andalusian Black, Andalusian Grey, Angeln, Angus, Ankole, Ankole-Watusi, Argentine Criollo, Asturian Mountain, Asturian Valley, Australian Braford, Australian Lowline, Ba-Bg, Bachaur, Baladi, Barka, Barzona, Bazadais, Beefalo, Beefmaker, Beefmaster, Belarus, Red, Belgian Blue, Belgian Red, Belmont Adaptaur, Belmont Red, Belted Galloway, Bengali, Berrendas, Bh-Bz, Bhagnari, Blanco Orejinegro, Blonde d'Aquitaine, Bonsmara, Boran, Braford, Brahman, Brahmousin, Brangus, Braunvieh, British White, Busa, Cachena, Canary Island, Canchim, Carinthian Blond, Caucasian, Channi, Charbray, Charolais, Chianina, Cholistani, Corriente, Costello con Cuernos, Dajal, Damietta, Dangi, Deoni, Devon, Dexter, Dhanni, Dolafe, Droughtmaster, Dulong, East Anatolian Red, Enderby Island, English Longhorn, Evolene, Fighting Bull, Florida Cracker/Pineywoods, Galician Blond, Galloway, Gaolao, Gascon, Gelbray, Gelbvieh, German Angus, German Red Pied, Gir, Glan, Greek Shorthorn, Guzerat, Hallikar, Hariana, Hays Converter, Hereford, Herens, Highland, Hinterwald, Holando-Argentino, Horro, Hungarian Grey, Indo-Brazilian, Irish Moiled, Israeli Red, Jamaica Black, Jamaica Red, Jaulan, Kangayam, Kankrej, Kazakh, Kenwariya, Kerry, Kherigarh, Khillari, Krishna Valley, Kurdi, Kuri, Limousin, Lincoln Red, Lohani, Luing, Maine Anjou, Malvi, Mandalong, Marchigiana, Masai, Mashona, Mewati, Mirandesa, Mongolian, Morucha, Murboden, Murray Grey, Nagori, N'dama, Nellore, Nguni, Nimari, Ongole, Orma Boran, Oropa, Parthenais, Philippine Native, Polish Red, Polled Hereford, Ponwar, Piedmontese, Pinzgauer, Qinchuan, Ratien Gray, Rath, Rathi, Red Angus, Red Brangus, Red Poll, Retinta, Rojhan, Romagnola, Romosinuano, RX3, Sa-Sg, Sahiwal, Salers, Salorn, Sanhe, Santa Cruz, Santa Gertrudis, San Martinero, Sarabi, Senepol, Sh-Sz, Sharabi, Shorthorn, Simbrah, Simmental, Siri, Slovenian Cika, South Devon, Sussex, Swedish Red Polled, Tarentaise, Telemark, Texas Longhorn, Texon, Tharparkar, Tswana, Tuli, Ukrainian Beef, Ukrainian Grey, Ukrainian Whitehead, Umblachery, Ural Black Pied, Vestland Red Polled, Vosges, Wagyu, Welsh Black, White Caceres, White Park, Xinjiang Brown and Yanbian cattle breeds, as well as animals bred therefrom and related thereto.

### II. Nucleic Acid Detection

Techniques for nucleic acid detection may find use in certain embodiments of the invention. For example, such techniques may find use in scoring individuals for genotypes or in the development of novel markers linked to the major effect locus identified herein.

1. Hybridization

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, nucleotide sequences may be used in accordance with the invention for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions may be used, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions may be rendered more stringent by the addition of increasing amounts of formamide.

For certain applications, lower stringency conditions may be preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M NaCl, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it may be advantageous to employ nucleic acids of defined sequences with the present invention in combination with an appropriate means, such as a label, for determining hybridization. For example, such techniques may be used for scoring of RFLP marker genotype. A wide variety of appropriate indicator means are known in the art, including fluorescent, radioactive, enzymatic, or other ligands, such as avidin/biotin, which are capable of being detected. In certain embodiments, one may desire to employ a fluorescent label or an enzyme tag such as urease, alkaline phosphatase, or peroxidase, instead of radioactive or other environmentally undesirable reagents. In the case of enzyme tags, colorimetric indicator substrates are known in the art that may be employed to provide a detection means that is visibly or spectrophotometrically detectable, to identify specific hybridization with complementary nucleic acid containing samples.

In general, it is envisioned that probes or primers may be useful as reagents in solution hybridization, as in PCR, for detection of nucleic acids, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481, and 5,919,626. Other methods of hybridization that may be used in the practice of the present invention are disclosed in U.S. Pat. Nos. 5,849,481, 5,849,486, and 5,851, 772. The relevant portions of these and other references identified in this section of the disclosure are incorporated herein by reference.

2. Amplification of Nucleic Acids

Nucleic acids used as a template for amplification may be isolated from cells, tissues or other samples according to standard methodologies. Such embodiments may find particular use with the invention, for example, in the detection of repeat length polymorphisms, such as microsatellite markers. In certain embodiments of the invention, amplification analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences may be employed. Primers may be provided in double-stranded and/or single-stranded form.

Pairs of primers designed to selectively hybridize to nucleic acids are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radio-label or fluorescent label or even via a system using electrical and/or thermal impulse signals. Typically, scoring of polymorphisms as fragment length variants will be done based on the size of the resulting amplification product.

A number of template-dependent processes are available to amplify oligonucleotide sequences present in a given template sample. One of the best known amplification methods is the polymerase chain reaction (referred to as PCR), which is well known in the art.

A reverse transcriptase PCR amplification procedure may be performed to obtain cDNA, which in turn may be scored for polymorphisms. Methods of reverse transcribing RNA into cDNA are well known. Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR and oligonucleotide ligase assay (OLA), disclosed in U.S. Pat. No. 5,912,148, also may be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291, and 5,942,391, GB Application No. 2 202 328, and in PCT Application No. PCT/US89/01025, each of which is incorporated herein by reference in its entirety.

An isothermal amplification method, in which restriction endonucleases and ligases are used to achieve the amplification of target molecules that contain nucleotide 5′-[alpha-thio]-triphosphates in one strand of a restriction site also may be useful in the amplification of nucleic acids in the present invention. Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; Gingeras et al., 1990; PCT Application WO 88/10315, incorporated herein by reference in their entirety). European Application No. 329 822 discloses a nucleic acid amplification process involving cyclically synthesizing single-stranded RNA (ssRNA), single-stranded DNA (ssDNA), and double-stranded DNA (dsDNA), which may be used in accordance with the present invention.

PCT Application No. WO 89/06700 (incorporated herein by reference in its entirety) discloses a nucleic acid sequence amplification scheme based on the hybridization of a promoter region/primer sequence to a target ssDNA followed by transcription of many RNA copies of the sequence. This scheme is not cyclic, i.e., new templates are not produced from the resultant RNA transcripts. Other amplification methods include “race” and “one-sided PCR.”

3. Detection of Nucleic Acids

Following any amplification, it may be desirable to separate the amplification product from the template and/or the excess primer. In one embodiment, amplification products are separated by agarose, agarose-acrylamide, or polyacrylamide gel electrophoresis using standard methods. Separated amplification products may be cut out and eluted from the gel for further manipulation. Using low melting point agarose gels, the separated band may be removed by heating the gel, followed by extraction of the nucleic acid.

Separation of nucleic acids also may be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography, as well as HPLC.

In certain embodiments, the amplification products may be visualized. A typical visualization method involves staining of a gel with ethidium bromide and visualization of bands under UV light. Alternatively, if the amplification products are integrally labeled with radio- or fluorometrically-labeled nucleotides, the separated amplification products may be exposed to x-ray film or visualized under the appropriate excitatory spectra.

In one embodiment, following separation of amplification products, a labeled nucleic acid probe is brought into contact with the amplified marker sequence. The probe may be conjugated to a chromophore or may be radiolabeled. In another embodiment, the probe may be conjugated to a binding partner, such as an antibody or biotin, or another binding partner carrying a detectable moiety.

In particular embodiments, detection may be by Southern blotting and hybridization with a labeled probe. The techniques involved in Southern blotting are well known to those of skill in the art. One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413, and 5,935,791, each of which is incorporated herein by reference.

4. Other Assays

Other methods for genetic screening may be used within the scope of the present invention, for example, to detect polymorphisms in genomic DNA, cDNA, and/or RNA samples. Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCR (see above), single-strand conformation polymorphism analysis (“SSCP”) and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA, or DNA/DNA molecule. This definition thus includes mismatches due to insertion/deletion mutations, as well as single or multiple base point mutations.

U.S. Pat. No. 4,946,773 describes an RNase A mismatch cleavage assay that involves annealing single-stranded DNA or RNA test samples to an RNA probe, and subsequent treatment of the nucleic acid duplexes with RNase A. For the detection of mismatches, the single-stranded products of the RNase A treatment, electrophoretically separated according to size, are compared to similarly treated control duplexes. Samples containing smaller fragments (cleavage products) not seen in the control duplex are scored as positive.

Other investigators have described the use of RNase I in mismatch assays. The use of RNase I for mismatch detection is described in literature from Promega Biotech. Promega markets a kit containing RNase I that is reported to cleave three out of four known mismatches. Others have described using the MutS protein or other DNA-repair enzymes for detection of single-base mismatches.

Alternative methods for detection of deletion, insertion or substitution mutations that may be used in the practice of the present invention are disclosed in U.S. Pat. Nos. 5,849,483, 5,851,770, 5,866,337, 5,925,525, and 5,928,870, each of which is incorporated herein by reference in its entirety.

5. Kits

All the essential materials and/or reagents required for screening cattle for a genetic marker genotype in accordance with the invention may be assembled together in a kit. This generally will comprise a probe or primers designed to hybridize specifically to individual nucleic acids of interest in the practice of the present invention, for example, primer sequences such as those for amplifying a SNP within or genetically linked to a hapblock set forth herein. Also included may be enzymes suitable for amplifying nucleic acids, including various polymerases (reverse transcriptase, Taq, etc.), deoxynucleotides, and buffers to provide the necessary reaction mixture for amplification. Such kits also may include enzymes and other reagents suitable for detection of specific nucleic acids or amplification products. Such kits generally will comprise, in suitable means, distinct containers for each individual reagent or enzyme as well as for each probe or primer pair.

### DEFINITIONS

The following definitions are provided to better define the present invention and to guide those of ordinary skill in the art in the practice of the present invention. Unless otherwise noted, terms are to be understood according to conventional usage by those of ordinary skill in the relevant art.

As used herein, the term “population” means a genetically heterogeneous group of animals that share a common parental derivation. A population in accordance with the invention may originate from a single breed or species of beef cattle, or may originate from multiple breeds or species of beef cattle.

As used herein, a “crossbred” or “hybrid” cattle refers to a head of cattle that resulted from a cross of one parent of one breed or species of cattle to another, distinct species or breed of cattle.

As used herein, “SNP” refers to single base positions in DNA at which different alleles, or alternative nucleotides, exist in a population, and are the most common form of genetic variation in the genome. The SNP position (interchangeably referred to herein as SNP, SNP site, SNP locus, SNP marker, biomarker, or marker) is usually preceded by and followed by highly conserved sequences of the allele (e.g., sequences that vary in less than 1/100 or 1/1000 members of the populations). An individual may be homozygous or heterozygous for an allele at each SNP position. In some embodiments, a SNP is referred to as a “cSNP” to denote that the nucleotide sequence containing the SNP is an amino acid coding sequence.

As used herein, references to SNPs and SNP genotypes include individual SNPs and/or haplotypes. Haplotypes may comprise groups of SNPs that are generally inherited together. Haplotypes can have stronger correlations with diseases or other phenotypic effects compared with individual SNPs, and therefore may provide increased diagnostic accuracy. Causative SNPs are those SNPs that produce alterations in gene expression or in the expression, structure, and/or function of a gene product, and therefore are most predictive of a possible clinical phenotype. One such SNP may be present within regions of genes encoding a polypeptide product, i.e. cSNPs. These SNPs may result in an alteration of the amino acid sequence of the polypeptide product (i.e., non-synonymous codon changes) and give rise to the expression of a defective or other variant protein. A SNP may also lead to premature termination of a polypeptide product. As referred to herein, causative SNPs may occur in any genetic region that can ultimately affect the expression, structure, and/or activity of a protein encoded by a nucleic acid. Such genetic regions may include, for example, those involved in transcription, such as SNPs in transcription factor binding domains, promoter regions, or areas involved in transcript processing, such as intron-exon boundaries, which may cause defective splicing, or SNPs in mRNA processing signal sequences such as polyadenylation signal regions. In another embodiment, a SNP may not be a causative SNP but may lie in close association with, and therefore segregate with, a sequence producing a trait or disease. In this situation, the presence of a SNP correlates with the presence of, or predisposition to, or an increased chance of the trait and may be useful for diagnostics, predisposition screening, and other uses.

Although the numerical chromosomal position of a SNP may still change upon annotating the current bovine genome, the SNP identification information such as variable alleles and flanking nucleotide sequences assigned to a SNP will remain the same. Genes discussed herein, as well as SNPs listed herein in Tables 6-8 are defined by a genomic position relative to the UMD 3.1 bovine genome assembly (Bos_{—}taurus_UMD_{—}3.1, GenBank assembly ID GCA_{—}000003055.3, Zimin et al., Genome Biology 10:R42, 2009). Those skilled in the art will readily recognize that the analysis of the nucleotides present in one or more SNPs set forth herein in an individual's genome can be done by any method or technique capable of determining nucleotides present in a polymorphic site using the published sequence information to the rs IDs or other SNP identification, such as KGP SNPs, for the SNPs listed herein. The nucleotides present in polymorphisms can be determined from either nucleic acid strand or from both strands.

As used herein, “gene” refers to a nucleic acid molecule that codes for a particular protein, or in certain cases, a functional or structural RNA molecule.

As used herein, a “haplotype” or “haplotype block” or “hapblock” refers to any combination of genetic markers (“alleles”). Such a haplotype may comprise one or more genes or markers conferring a desired trait. In some embodiments, such terms may refer to a set or collection of genes, SNPs, or other types of markers that are associated statistically. A haplotype can include two or more alleles and the length of a genome region including a haplotype may vary from a few hundred bases up to thousands of kilobases or megabases. As it is recognized by those skilled in the art, the same haplotype can be described differently by determining the haplotype defining alleles from different nucleic acid strands. The haplotypes described herein are differentially present in individuals with increased feed efficiency. Therefore, these haplotypes have diagnostic value. Detection of haplotypes can be accomplished by methods known in the art used for detecting nucleotides at polymorphic sites. The haplotypes described herein, e.g., having markers or SNPs set forth herein, are found more frequently in cattle with increased feed efficiency than in individuals with less feed efficiency. Alternatively, certain haplotypes may be found more frequently in cattle with less feed efficiency.

As used herein, an “allele” refers to one of two or more alternative forms of a genomic sequence at a given locus on a chromosome.

A “Quantitative Trait Locus (QTL)” is a chromosomal location that encodes for alleles that affect the expressivity of a phenotype.

As used herein, a “marker” means a detectable characteristic that can be used to discriminate between organisms. Examples of such characteristics include, but are not limited to, genetic markers, biochemical markers, metabolites, morphological characteristics, and agronomic characteristics.

As used herein, the term “phenotype” means the detectable characteristics of a cell or organism that can be influenced by gene expression.

As used herein, the term “genotype” means the specific allelic makeup of an animal.

As used herein, the term “introgressed” or “introduced,” when used in reference to a genetic locus or haplotype, refers to a genetic locus that has been introduced into a new genetic background. Introgression of a genetic locus can thus be achieved through breeding methods.

As used herein, the term “linked,” when used in the context of nucleic acid markers and/or genomic regions, means that the markers and/or genomic regions are located on the same linkage group or chromosome such that they tend to segregate together at meiosis.

As used herein, an “estimated breeding value” is a statistical numerical prediction of the relative genetic value of a particular individual for breeding. In one embodiment of the invention, an individual may be selected for breeding based upon its estimated breeding value. In particular, an individual with a desirable estimated breeding value may be selected.

In certain embodiments, a desirable estimated breeding value may refer to an estimated breeding value that is greater than the average estimated breeding value of the population of individuals being selected from. For instance, a desired estimated breeding value may be within the top 50%, 40%, 30%, 25%, 20%, 15%, 10%, 5%, 4%, 3%, 2%, or 1% of the population from which the individual is selected.

As used herein, the term “denoting” when used in reference to a genotype refers to any method whereby an animal or individual is indicated to have a certain genotype. This includes any means of identification of an individual having a certain genotype. Indication of a certain genotype may include, but is not limited to, any entry into any type of written or electronic medium or database whereby the individual's genotype is provided. Indications of a certain genotype may also include, but are not limited to, any method where an individual is physically marked or tagged. Illustrative examples of physical marking or tags useful in the invention include, but are not limited to, a barcode, a radio-frequency identification (RFID), a label, or the like.

As used herein, the singular forms “a,” “an,” “and,” and “the” include plural referents unless the context clearly dictates otherwise. Thus, for example, reference to “a SNP” includes a plurality of SNPs (i.e., at least one SNP), reference to “a gene” includes a plurality of genes (i.e., at least one gene), and so forth.

### EXAMPLES

The following disclosed embodiments are merely representative of the invention which may be embodied in various forms. Thus, specific structural, functional, and procedural details disclosed in the following examples are not to be interpreted as limiting.

### Example 1

### Feed Efficiency Phenotype

For this study, 174 Nellore-Angus F2 steers in 13 full-sibling families, produced by embryo transfer, from the Texas A&M McGregor Genomics research population located in central Texas (latitude: 31.3865, longitude: −97.4105) were utilized. Calves were produced in the spring and fall calving seasons, and this study used calves born from 2003 to 2005. After weaning (approximately 230 d of age) the animals were grass fed for approximately 130 d until they reached 11 to 13 mo of age. Steers were moved to a Calan Broadbent Feeding System (American Calan; Northwood, N.H.) to enable measurement of individual feed intake. Over 28 d, the steers were adjusted to the finishing diet (Table 1). Feeding was ad libitum and uneaten food was removed and measured every 7 d. Animals were weighed every 28 d for ˜140 d at the same time of day and in the same order of pens at each weigh day to equalize gut fill effects across time, as much as possible.

Using the National Research Council (NRC, Nutrient Requirements of Beef Cattle: Seventh Revised Edition: Update 2000. Washington, D.C.: The National Academies Press, 2000) model application, daily feed intake required to achieve observed average daily gain (ADG) was predicted. Model inputs included feeding period mid-weight (used as the current reference weight for model predictions) and final weight at slaughter for each animal. Standardized inputs were used for animal type (growing/finishing), age (14 mo), sex (steer), BCS (5), breed (Nellore-Angus 2-way cross), management (ionophore), diet (Table 1) and grading system (select). Environmental factors in the model were set to be thermoneutral. Model predicted intake was subtracted from observed dry matter intake (DMI) and the difference was defined as residual feed intake based on the NRC model (RFINRC), such that those animals that consumed less than predicted (and thus, were more efficient) had negative RFINRC (Liu et al, 2000). This method of residual prediction parallels more typical calculations of residual feed intake (RFI), in which individual residuals represent deviation from a modeled mean intake at an observed rate of gain. However, the standard method is generally restricted to use within a single contemporary group fed simultaneously, or using contemporary group as a model effect. In this case, a standard model (the NRC) was utilized to construct the modeled mean intake rather than a regression on observed data, allowing animals in multiple contemporary groups to be evaluated against a common model.

TABLE 1
Ration formulation

| Ingredient | %¹ |
|---|---|
| Ground milo | 20.00 |
| Ground corn | 31.25 |
| Cottonseed meal | 9.00 |
| Cottonseed hulls | 25.00 |
| Molasses | 10.00 |
| Premix² | 3.00 |
| Ammonium chloride | 0.25 |
| R-1500³ | 1.50 |

¹Expressed as a percent on an as-fed basis.
²Composition of premix: ground limestone, 60%; trace mineralized salt, 16.7% (NaCl, 98%; Zn, 0.35%; Mn, 0.28%; Fe, 0.175%; Cu, 0.035%; I, 0.007%; Co, 0.007%); mono-dicalcium phosphate, 13%; potassium chloride, 6.7%; Vitamin premix, 3.3% (vitamin A, 2,200,000 IU/kg; vitamin D, 1,100,000 IU/kg, vitamin E, 2,200 IU/kg); Zinc oxide, 0.33%.
³R-1500 contains 1.65 g monensin sodium (Rumensin ™) per kg.

### Example 2

### Sample Selection

For gene expression analysis, 36 animals were identified at the tails of the efficiency distribution based on RFINRC as described above. A total of 18 animals were classified as most “efficient” for this population and had negative RFINRC residuals, indicating that they had consumed less feed than would be expected based on the model. A total of 18 animals were classified as most “inefficient” with positive RFINRC residuals, indicating they had consumed more feed than would be expected based on the model. Muscle samples from these 36 animals were used for subsequent expression analysis. A statistically average group of 18 animals with an RFINRC residual clustered around zero was added for comparison purposes. Thus, a total of 54 animals from the middle and both tails of the residual distribution were analyzed for gene expression. Means for these groups are presented in Table 2.

TABLE 2
Simple means (±std err) for RFI residuals by efficiency groups

| Item | Efficient | Average | Inefficient |
|---|---|---|---|
| n | 18 | 18 | 18 |
| RFI residuals | −2.3 ± 0.1^{a} | 0.0 ± 0.0^{b} | 2.3 ± 0.1^{c} |

^{a,}^{b,}^{c}Means within a row with different superscripts differ significantly (P < 0.01).

### Example 3

### Tissue Collection and Extraction of RNA

Steers were harvested at 18 mo of age at the Rosenthal Meat Center at Texas A&M University in College Station, Tex., using humane harvesting procedures, as described by Savell and Smith (2000). Animals were restricted from feed for approximately 12 hr before harvest, but had continual access to water. Animals were immobilized using a captive bolt stunning mechanism and further processed using standard industry procedures. Approximately 1 g of muscle tissue from the Longissimus cervicis (in the neck region of the carcass) was collected shortly after death and before electrical stimulation (ES), less than 1 hr post-exsanguination of the carcass. The muscle sample was flash frozen in liquid nitrogen to prevent mRNA degradation. Samples were stored at −80° C. until RNA was extracted.

Total RNA was extracted from approximately 100 to 200 mg of whole muscle tissue (L. cervicis) from each of the 54 animals with TRI Reagent® (Molecular Research Center, Cincinnati, Ohio) and 1-bromo-3-chloropropane (Molecular Research Center). Next, RNA was precipitated with isopropanol (Sigma Aldrich, St. Louis, Mo.), washed with 70% ethanol (Sigma Aldrich), and reconstituted in 50 μL nuclease-free water (Invitrogen, Carlsbad, Calif.). Quality of the RNA was assessed on an Agilent 2100 series Bioanalyzer (Agilent Technologies, Palo Alto, Calif.) according to the manufacturer's protocol. Samples with an RNA integrity number (RIN)>8.0 and an appropriate electropherogram image were treated with DNase (Invitrogen) and column-purified via RNeasy Mini kit (Qiagen, Valencia, Calif.). RNA extracts were stored at −80° C. prior to microarray labeling.

### Example 4

### Microarray

Twenty-four samples representing the animals at the most extreme tails of the efficiency distribution were labeled for microarray analysis using a 2-color microarray-based gene expression analysis. Total RNA (2.5 μg) was labeled with cyanine 3-CTP or cyanine 5-CTP (Quick Amp Labeling Kit, Agilent Technologies, Santa Clara, Calif.) according to the manufacturer's protocol. Labeled cRNA (750 ng of each sample) was hybridized to bovine 4×44K stock arrays (Agilent array #015344) at 65° C. for 17 h in an Agilent microarray hybridization chamber. Arrays were washed according to the manufacturer's instructions, dried by brief submersion in Agilent's Drying & Stabilization solution, and scanned immediately on an Agilent G2505 C scanner at 5 μm resolution with extended dynamic range (passes at 100% and 10% PMT).

Normalization and quality control were performed using embedded functions in Agilent Genespring (v10.0.2) software. Array data were subjected to baseline normalization to the median expression level and had a minimum threshold of 1.0 expression, all using standard quality control options included in Genespring software. Following baseline normalization and QC, genes of interest were selected based on relative expression differences. A false discovery rate calculation was not used, as these samples failed the assumption of independence. These microarray data were deposited in NCBI's Gene Expression Omnibus (Edgar et al., 2002) and are accessible through GEO Series accession number GSE56705.

Microarray analysis yielded 58 genes for which relative expression was significantly different between efficiency groups, and the difference between groups was 1.5-fold or greater. The ACTN3 gene was identified as more greatly expressed in the inefficient group (P=0.051) of steers. Because different alleles of this gene have been associated with differential athletic performance in humans, and ACTN3 genotype is thought to contribute to variation in muscle phenotype (North et al., 2003; Yang et al., 2003), ACTN3 was selected for further investigation in this study. The ACTN2 gene was selected for analysis because of its complementary role to ACTN3. The RPS20, COX7C, and COX3 genes were selected as possible control reference genes based on the literature. The genes, ADFP, ATP5B, HKII, and LDHB were all selected as genes that play a role in muscle metabolism. CAPN1, CAPN2, and CAST were examined because of their roles in muscle turnover, and MYH1 and MYH2 were selected for their roles in muscle growth. Initially COX3 and COX7 were tested as potential reference genes for normalization because they were previously observed to exhibit stable expression in bovine liver (Kochan et al., 2009). However, as might be expected from skeletal muscle, expression of COX3 and COX7 was not stable across these samples, and were not useful as reference genes. Instead, RPS20 was used as a reference for normalization due to a low degree of variance in expression among groups.

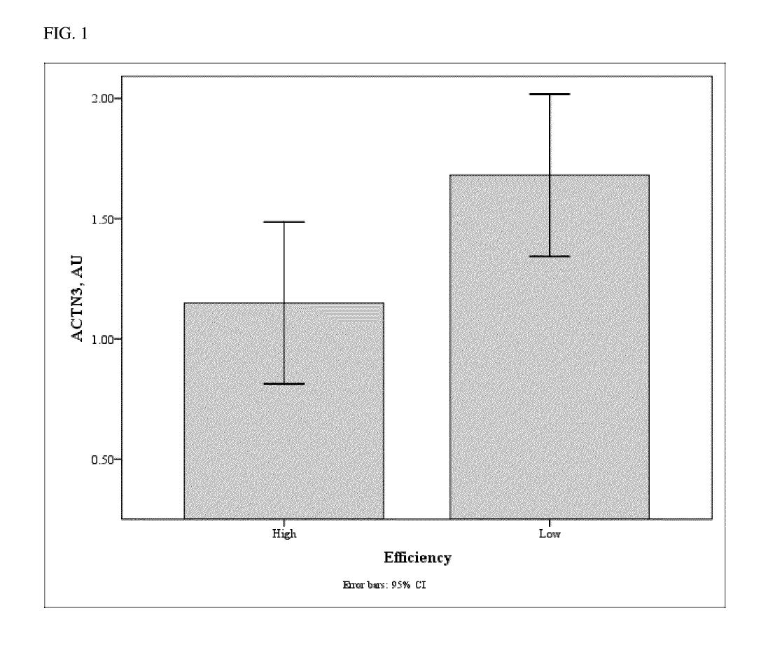

By qRT-PCR, expression of ACTN3 was 1.6-fold greater (P=0.009) in the average and inefficient groups than in the efficient group (FIG. 1 and Table 3). ACTN2 expression did not vary significantly between any of the groups, nor did it correlate significantly with ACTN3 expression or fiber type ratio. Of the other genes assayed, LDHB expression was significantly lower in the average group but not the inefficient group, relative to the efficient steers. The mRNA quantity of the remaining genes examined (Table 3) did not differ significantly between groups.

TABLE 3
Relative mRNA expression of selected genes by qRT-PCR analysis

Classification

| Item | Average/Efficient | Inefficient/Efficient |
|---|---|---|
| N | 18 | 18 |
| ACTN3 | 1.6 + 0.06^{a} | 1.6 + 0.05^{a} |
| ACTN2 | 1.2 + 0.05 | 1.0 + 0.05 |
| COX3 | 1.7 + 0.05^{a} | 1.4 + 0.06^{b} |
| COX7C | 1.1 + 0.03 | 0.9 + 0.04 |
| HKII | 0.9 + 0.04 | 0.8 + 0.05 |
| LDHB | 0.6 + 0.03^{a} | 0.9 + 0.04^{b} |
| PRDX3 | 0.9 + 0.03 | 1.0 + 0.04 |

Relative expression for the average and inefficient groups is calculated and presented as a fold-ratio compared to the efficient group. Expression was normalized to RPS20.
^{a,}^{b}Means with subscripts differ from efficient group P < 0.05. Means with different subscripts differ from each other P < 0.05.

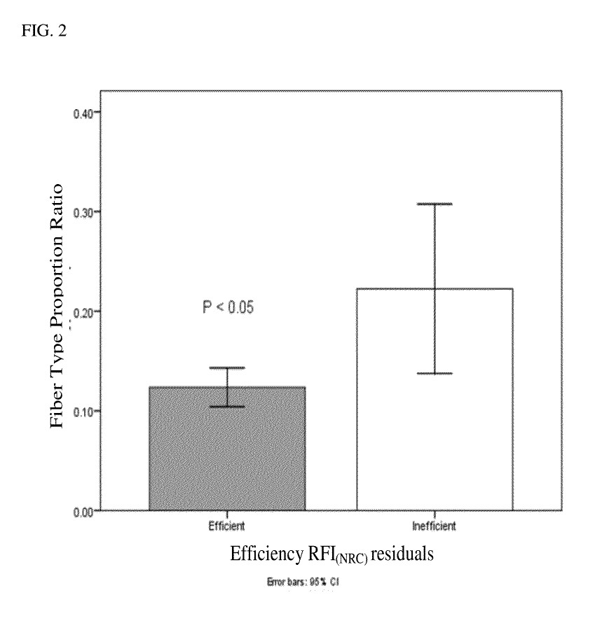

Post-transcriptional modifications, other regulatory mechanisms, and differences in timing of expression can make correlation of mRNA expression with protein expression difficult (Greenbaum et al., 2002). To verify that the observed difference in ACTN3 gene expression translated to actual differences in muscle protein expression, 12 samples (portions of the same samples used for qRT-PCR) from each extreme tail of the distribution (n=24) were assayed for fiber type ratios based on gel separation of muscle fiber type specific isoforms. The ratio of fast-twitch to slow-twitch muscle fiber (Type IIx/Type I) was 1.8-fold higher (P=0.027) in the inefficient group compared to the efficient group (FIG. 2).

### Example 5

### Real-Time Quantitative Reverse Transcriptase Polymerase Chain Reaction

Quantitative RT-PCR analysis was conducted on the full set of 54 samples. Synthesis of cDNA from all RNA samples was accomplished with the High-Capacity cDNA Reverse Transcription Kit (Invitrogen) with a starting quantity of 2 μg mRNA per 40 μL reaction. Oligo(dT)20 primers (Integrated DNA Technologies, Coralville, Iowa), 5 μM final concentration, were used for reverse transcription. cDNA was diluted 1:2 in yeast tRNA (25 ng/μl; Invitrogen). Samples were amplified in a total volume of 20 μL containing 1×SYBR GreenER™ qPCR SuperMix (Invitrogen), 300 nM primers, and 2 μL template cDNA. Real-time quantitative RT-PCR (qRT-PCR) was performed at 95° C. for 10 min followed by 40 cycles of 15 s at 95° C. and 60 s at 60° C., in a 7900HT thermal cycler (Applied Biosystems). Amplification data were analyzed with the Sequence Detection System v2.2.2 program (Applied Biosystems). Expression was normalized to RPS20 as a reference gene (de Jonge et al., 2007), and relative expression was quantified by the AACt method (Livak and Schmittgen, 2001).

Primers for genes validated by qRT-PCR were designed using Oligo 6 Primer Analysis Software v6.71 (Molecular Biology Insights, Cascade, Colo.), and several potential reference genes were also evaluated. Genes for further analysis were identified based on differential expression in the microarray study, and candidate reference genes. Genes analyzed included actinin 2 (ACTN2), actinin 3 (ACTN3), ribosomal protein S20 (RPS20), cytochrome c oxidase subunit 7C (COX7C), cytochrome c oxidase III (COX3), adipose differentiation-related protein (ADFP), ATP synthase H+transporting, mitochondrial F1 complex, beta polypeptide (ATP5B), hexokinase 2 (HKII), and lactate dehydrogenase B (LDHB). Genes and primers used are presented in Table 4. Primer pairs were evaluated by BLAST sequence similarity search. Primer pairs were selected that did not cross-amplify across species or between different mRNA transcripts. Additionally, primers were selected to span an exon junction to prevent genomic amplification.

TABLE 4
Complete list of primer pairs used for qRT-PCR assays and their
function in muscle. SEQ ID NOs are listed in parentheses.

| Gene | | |
|---|---|---|
| Symbol | Description | Sequence¹ (SEQ ID NO) |
| ACTN2 | actinin, alpha 2 | GGTCTTTGACAACAAGCA (1) |
| | | TGATGGTTCTGGCGATA (2) |
| ACTN3 | actinin, alpha 3 | CGGGAGACAAGAACTACATCA (3) |
| | | CGTAGAGGGCACTGGAGAA (4) |
| ATP5B | ATP synthase, H+ transporting, | CCCATCAAAACCAAGCAA (5) |
| | mitochondrial F1 complex, | TCAACACTCATCTCCACGAA (6) |
| | beta polypeptide | |
| CAPN1 | calpain 1, (mu/I) large subunit | GACCATAGGCTTCGCTGTCT (7) |
| | | AGGTTGATGAACTGCTCGGA (8) |
| CAPN2 | calpain 2, (m/II) large subunit. | CGACTGGAGACACTGTTCAGGA (9) |
| | | CTTCAGGCAGATTGGTTATCACTT (10) |
| CAST | calpastatin | GCTGTCGTCTCTGAAGTGGTT (11) |
| | | GGCATCGTCAAGTTCTTTGTTGT (12) |
| COX3 | mitochondrially encoded | CCACCACTTCGGCTTTGAAG (13) |
| | cytochrome c oxidase III | GGAAAAGTCAGACTACGTCTACGAAA (14) |
| COX7C | Cytochrome c oxidase | TGCAGCCGCCATTTCTTC (15) |
| | subunit VIIa | TAGCGCTGTTGGACGCTCTA (16) |
| HKII | hexokinase 2 | TCAACACTCATCTCCACGAA (17) |
| | | CACCACAGCAACCACATC (18) |
| LDHB | lactate dehydrogenase B | CAGAAATGGGAACAGACAA (19) |
| | | GACTTCATAGGCACTCTCAAC (20) |
| MYH1 | myosin, heavy chain 1, skeletal | TGAGGAAGCGGAGGAACAAT (21) |
| | muscle, adult | TGGGACTCGGCAATGTCA (22) |
| MYH2 | heavy chain 2, skeletal | CAATGACCTGACAACCCAGA (23) |
| | muscle, adult | CCTTGACAACTGAGACACCAGA (24) |
| RPS20 | ribosomal protein S20 | ACCAGCCGCAACGTGAA (25) |
| | | CCTTCGCGCCTCTGATCA (26) |

¹COX3, COX7C, and RPS20 primer sequences provided by Kochen, 2009.

SDS (v.2.4) software from Applied Biosystems (Carlsbad, Calif.) was used to quantify qRT-PCR results. A threshold value of 0.20 was used to determine Ct value. Relative expression was calculated using the method described by Livak and Schmittgen (2001). In summary, raw Ct values were normalized to RPS20 based on internal expression stability between groups (Vandesompele et al., 2002). The normalized value was subtracted from the raw Ct for each sample (ACT). From the ACT the average value of the efficient group was subtracted (AACT). The AACT was linearized by taking the inverse negative log-base 2 (RQ). The efficient group in this study will thus always have a median expression value of 1.0. It should be noted that these are arbitrary units (AU) and that no direct comparison between different genes in total expression levels can be reliably made. All values are relative and applicable directly only as a within-group comparison of relative expression.

By qRT-PCR analysis, it was confirmed that differences in ACTN3 expression in skeletal muscle samples existed between the high and low feed efficiency groups of steers (P=0.009). Expression of ACTN2 did not differ significantly between groups. Relative to the efficient group, the inefficient group overexpressed ACTN3 1.6-fold. The fiber type ratio measured by fast-twitch (Type IIx)/slow-twitch (Type I) differed between groups (P=0.027), with a 1.8-fold increase in the inefficient group relative to the efficient group. As ACTN3 is expressed only in Type II fibers, the fiber type data are consistent with expression data. The inefficient group also had a larger standard error than the efficient group.

### Example 6

### Muscle Fiber Type Classification

Fiber type analysis was determined by gel electrophoresis. L. cervicis muscle samples (0.1 g) were homogenized in 500 μL of buffer containing 250 mM sucrose, 100 mM KCl, 5 mM EDTA and 20 mM Tris-HCl, pH 6.8. The homogenate was filtered through nylon cloth to remove debris and centrifuged at 10,000×g for 10 min. The pellet obtained was re-suspended in a 500 μL of washing buffer containing 200 mM KCl, 5 mM EDTA, 0.5% Triton X-100 and 20 mM Tris-HCl, pH 6.8. The suspension was centrifuged at 10,000×g for 10 min. The pellet containing purified myofibrillar proteins was resuspended in 200 μL water and 300 μL of standard 2× sample loading buffer, boiled for 5 min then centrifuged at 12,000×g for 5 min. The resultant supernatant was used for electrophoresis.

The stacking gels consisted of 4% acrylamide (acrylamide: bis=50:1) and 5% (v/v) glycerol, 70 mM Tris-HCl, pH 6.7, 0.4% (w/v) SDS, 4 mM EDTA, 0.1% (w/v) APS, and 0.01% (v/v) TEMED. The separation gel contained 5% (w/v) glycerol, acrylamide: bis (50:1) at a concentration ranging from 5 to 20%, 200 mM Tris, pH 8.8, 4 mM EDTA, 0.4% (w/v) SDS, 0.01% (v/v) TEMED, and 0.1% (w/v) ammonium persulfate. The upper running buffer consisted of 0.1 M Tris-HCl, pH 8.8, 0.1% (w/v) SDS, 150 mM glycine, and 10 mM mercaptoethanol, and the lower running buffer consisted of 50 mM Tris-HCl, pH 8.8, 0.01% (w/v) SDS, and 75 mM glycine. Gels were run at 8° C. in a Hoefer SE 600 (Hoefer Scientific, San Francisco, Calif.) unit, at constant 200 V for 24 h (Bamman et al., 1999). After electrophoresis, gels were stained with Coomassie blue and scanned with a densitometer to determine the amount of each myosin isoform and percentage of Type I and Type IIx muscle fibers (Underwood et al., 2007).

### Example 7

### Haplotype Analysis

All individuals (n=776) in the first three generations of the experimental population were previously genotyped with the Illumina BovSNP50 v1 assay (Illumina, Inc., San Diego, Calif.), and data were available for use in this study. After pruning genotypes to remove animals with poor completion rate (<0.9), SNPs with low minor allele frequency (<0.05), SNPs with poor completion rates (<0.9), and those SNPs that deviated from Hardy-Weinberg equilibrium (P<0.0001), 39,890 genotypes per individual remained. To determine whether breed of origin or parent of origin played a role in the efficiency phenotype, SNP genotypes spanning 1-Mb intervals flanking several genes of interest (Table 5) based on expression analyses were extracted using PLINK v1.07 (Purcell et al., 2007) and formatted for phase v2.1.1 software (Stephens et al., 2001). Haplotypes were established using 100 iterations of phase v2.1.1, with a thinning interval of 2 and a burn-in of 10. Resultant phased haplotypes were ordered by generation, and breed (Nellore or Angus) and parent of origin were manually tracked through the pedigree to assign the source of each haplotype block in the F2 generation. Since gene expression analyses of calpain-1 (CAPN1), calpain-2 (CAPN2), calpastatin (CAST), myosin heavy chain 1 (MYH1), and myosin heavy chain 2 (MYH2) genes were also available from another study (Vaughn, 2013), their relationship with feed efficiency was also examined.

TABLE 5
SNP haplotype block location information

| | | | | Number of |
|---|---|---|---|---|
| | | | | informative SNP |
| Gene | Chromosome | Coordinate (UMD 3.1) | | in 1 Mb region |
| CAPN1 | 29 | 44063463 | 44113492 | 24 |
| CAPN2 | 16 | 27781671 | 27840011 | 24 |
| CAPN3 | 10 | 37829007 | 37885645 | 24 |
| CAST | 7 | 98444979 | 98581253 | 18 |
| ACTN2 | 28 | 9403203 | 9450920 | 16 |
| ACTN3 | 29 | 45230630 | 45242406 | 16 |
| MYH1/2 | 19 | 30110728 | 30165109 | 18 |
| FN1 | 2 | 103881402 | 103950562 | 12 |

Coordinates are based on the UMD 3.1 bovine genome assembly (Bos_taurus_UMD_3.1, GenBank assembly ID GCA_000003055.3).

Haplotype block analysis was conducted for ACTN2, ACTN3, CAPN1, CAPN2, CAPN3, CAST, FN1, and MYH1/2. Three of the haplotype blocks were associated with significant differences in efficiency between Angus and Nellore in the larger population studied. The CAPN2 and ACTN3 Nellore haplotype blocks were associated with superior efficiency when inherited maternally (P=0.03). SNPs corresponding to the hapblocks for ACTN3, FN1, and CAPN2 are provided in Tables 6-8. A role for ACTN3 expression in influencing bovine metabolic efficiency has not been previously reported. The FN1 Angus haplotype block was associated with superior efficiency when inherited maternally (P=0.04). Neither the CAST nor the CAPN1 haplotypes were associated with any improvement in efficiency (Table 9).

TABLE 6
SNPs corresponding to ACTN3 hapblock on bovine chromosome 29

| SNP ID | Position |
|---|---|
| ARS-BFGL-NGS-101448 | 44740917 |
| ARS-USMARC-Parent-DQ404153-no-rs | 44756502 |
| ARS-BFGL-NGS-27390 | 44769768 |
| UA-IFASA-5680 | 44807928 |
| BTB-01033776 | 44853970 |
| BTA-66033-no-rs | 44900940 |
| ARS-BFGL-NGS-60670 | 44940036 |
| ARS-BFGL-NGS-119847 | 44969518 |
| ARS-BFGL-NGS-20927 | 44979377 |
| ARS-BFGL-NGS-34609 | 44999264 |
| ARS-BFGL-NGS-109360 | 45023665 |
| ARS-BFGL-NGS-103780 | 45102557 |
| ARS-BFGL-NGS-104441 | 45129099 |
| Hapmap30072-BTA-65952 | 45166442 |
| ARS-BFGL-NGS-41686 | 45187114 |
| ARS-BFGL-NGS-35005* | 45210680 |
| ARS-BFGL-NGS-29040* | 45287502 |
| BTB-01033227 | 45326585 |
| ARS-BFGL-NGS-14337 | 45367095 |
| Hapmap41926-BTA-65940 | 45458280 |
| Hapmap53679-rs29025626 | 45482143 |
| ARS-BFGL-NGS-108350 | 45504697 |
| ARS-BFGL-NGS-3727 | 45530264 |
| ARS-BFGL-NGS-35555 | 45575574 |
| ARS-BFGL-NGS-56568 | 45660037 |
| ARS-BFGL-NGS-23884 | 45686541 |

*Indicates SNPs that flank the ACTN3 gene.

TABLE 7
SNPs corresponding to FN1 hapblock on bovine chromosome 2

| SNP ID | Position |
|---|---|
| ARS-BFGL-NGS-96747 | 103421027 |
| BTA-48534-no-rs | 103470685 |
| Hapmap49191-BTA-48538 | 103491995 |
| ARS-BFGL-NGS-3072 | 103528661 |
| BTA-05667-rs29019825 | 103592247 |
| ARS-BFGL-BAC-5705 | 103707675 |
| ARS-BFGL-BAC-35929 | 103731623 |
| Hapmap26477-BTA-161779 | 103810327 |
| ARS-BFGL-NGS-117549* | 103864593 |
| ARS-BFGL-BAC-35928* | 103890577 |
| UA-IFASA-6155* | 103916046 |
| ARS-BFGL-NGS-4334* | 103947921 |
| BTA-48084-no-rs | 103995601 |
| ARS-BFGL-NGS-23725 | 104026376 |
| Hapmap50004-BTA-48076 | 104083149 |
| Hapmap33475-BTA-134171 | 104123985 |
| ARS-BFGL-BAC-35925 | 104145868 |
| BTA-97778-no-rs | 104256806 |
| ARS-BFGL-NGS-30643 | 104297699 |
| Hapmap43422-BTA-97780 | 104320886 |
| ARS-BFGL-NGS-75769 | 104349734 |

*Indicates SNPs that fall within or flank the FN1 gene.

TABLE 8
SNPs corresponding to CAPN2 hapblock on bovine chromosome 16

| SNP ID | Position |
|---|---|
| ARS-BFGL-NGS-79492 | 27277052 |
| ARS-BFGL-NGS-1328 | 27334730 |
| Hapmap30214-BTA-130514 | 27401728 |
| ARS-BFGL-NGS-117377 | 27432134 |
| Hapmap26112-BTA-130531 | 27466587 |
| ARS-BFGL-NGS-13101 | 27489237 |
| Hapmap48734-BTA-38315 | 27527228 |
| ARS-BFGL-NGS-54368 | 27572128 |
| ARS-BFGL-NGS-56645 | 27602073 |
| ARS-BFGL-NGS-39696 | 27629566 |
| ARS-BFGL-NGS-38652 | 27671754 |
| ARS-BFGL-NGS-60495 | 27692042 |
| ARS-BFGL-NGS-80292 | 27720210 |
| ARS-BFGL-NGS-12538 | 27752636 |
| ARS-BFGL-NGS-102216* | 27805751 |
| ARS-BFGL-NGS-85727* | 27833776 |
| Hapmap30619-BTA-38329 | 27894801 |
| BTA-38323-no-rs | 27929423 |
| ARS-BFGL-NGS-62655 | 27978288 |
| Hapmap60671-rs29023031 | 28006296 |
| BTA-38222-no-rs | 28042634 |
| ARS-BFGL-NGS-80253 | 28155110 |
| ARS-BFGL-NGS-86281 | 28295660 |

*Indicates SNPs that fall within the CAPN2 gene.

TABLE 9
Efficiency statistics based on haplotype block analysis

| Haplotype block | | | | | | Paternal | | Maternal | |
|---|---|---|---|---|---|---|---|---|---|
| | | NN | NA | AN | AA | NN/NA | AN/AA | NN/AN | NA/AA |
| ACTN3 | n | 41 | 49 | 40 | 44 | 90 | 84 | 81 | 93 |
| | RFI res. | −0.7 ± 0.5 | 0.5 ± 0.4 | −0.3 ± 0.4 | 0.5 ± 0.4 | 0.0 ± 0.3 | 0.1 ± 0.3 | −0.5^{a} ± 0.3 | 0.5^{b} ± 0.3 |
| CAST | n | 46 | 43 | 42 | 43 | 89 | 85 | 88 | 86 |
| | RFI res. | 0.5 ± 0.4 | −0.8 ± 0.4 | 0.2 ± 0.3 | 0.2 ± 0.5 | −0.1 ± 0.3 | 0.2 ± 0.3 | 0.4 ± 0.3 | −0.3 ± 0.3 |
| CAPN1 | n | 23 | 39 | 37 | 75 | 62 | 112 | 60 | 114 |
| | RFI res. | −0.6 ± 0.6 | 0.4 ± 0.4 | −0.3 ± 0.4 | 0.2 ± 0.4 | 0.0 ± 0.3 | 0.0 ± 0.3 | −0.4 ± 0.3 | 0.3 ± 0.3 |
| CAPN2 | n | 41 | 52 | 40 | 41 | 93 | 81 | 81 | 93 |
| | RFI res. | −0.7 ± 0.5 | 0.5 ± 0.4 | −0.3 ± 0.4 | 0.4 ± 0.5 | 0.0 ± 0.3 | 0.1 ± 0.3 | −0.5^{a} ± 0.3 | 0.5^{b} ± 0.3 |
| FN1 | n | 59 | 52 | 32 | 31 | 111 | 63 | 91 | 83 |
| | RFI res. | 0.4 ± 0.3 | −0.5 ± 0.5 | 0.6 ± 0.5 | −0.4 ± 0.5 | 0.0 ± 0.3 | 0.1 ± 0.3 | 0.5^{a} ± 0.3 | −0.4^{b} ± 0.3 |

^{a,b}Means within a row with different superscripts differ significantly (P < 0.05).

Among all steers with records, from which a subset was used for gene expression assays, parent and breed of origin of the ACTN3 haplotype block had a significant impact on efficiency as measured by RFI residuals. The Nellore haplotype block was associated with greater efficiency when inherited maternally (−0.47 compared to 0.49, P=0.03) regardless of the breed of origin of the paternal haplotype block. Additionally, the Nellore haplotype blocks of CAPN2 and FN1 were associated with differences in efficiency when inherited maternally. For CAPN2 a maternally inherited Nellore haplotype was linked to improved efficiency (−0.50 compared to 0.50, P=0.03). The FN1 Angus haplotype block was associated with improved efficiency relative to the Nellore (−0.40 compared to 0.50, P=0.04). No paternal haplotype blocks were implicated to play a role in this trait.

### Example 8

### Statistical Analysis

SPSS 16.0 software (IBM, Armonk, N.Y.) was used for all statistical analyses. To test for significance between efficiency groups, an independent samples t-test was used. Correlation analysis used a bivariate two-tailed Pearson's correlation. All qRT-PCR expression was normalized to the RPS20 reference gene (de Jonge et al., 2007). Expression data are reported relative to the feed efficient group, which was set to a value of 1.0. The inefficient and average groups are represented as fold change relative to the efficient group. One sample was removed from all analysis because expression values measured for the reference gene, RPS20, were not consistent with other samples.

What is claimed is:

1. A method of selecting a head of beef cattle with a genetic predisposition for increased feed efficiency comprising selecting said head of beef cattle based on the presence in the genome of at least one genetic haplotype conferring said increased feed efficiency selected from the group consisting of:
(a) a haplotype located in a region on bovine chromosome 29 defined by positions 45230630 and 45242406;
(b) a haplotype located in a region on bovine chromosome 2 defined by positions 103881402 and 103950562; and
(c) a haplotype located in a region on bovine chromosome 16 defined by positions 27781671 and 27840011.

2. The method of claim 1, comprising selecting said head of beef cattle based on the presence in the genome of at least two of said haplotypes.

3. The method of claim 1, wherein said haplotype of (a) is inherited from the maternal parent.

4. The method of claim 1, wherein said haplotype of (a) is inherited from the paternal parent.

5. The method of claim 1, wherein said haplotype of (a) is inherited from the genome of a Nellore head of cattle.

6. The method of claim 1, wherein said haplotype of (b) is inherited from the paternal parent.

7. The method of claim 1, wherein said haplotype of (b) is inherited from the genome of an Angus head of cattle.

8. The method of claim 1, wherein said haplotype of (c) is inherited from the maternal parent.

9. The method of claim 1, wherein said haplotype of (c) is inherited from the genome of a Nellore head of cattle.

10. The method of claim 1, further comprising sequencing said haplotype.

11. The method of claim 1, further comprising detecting at least one SNP within or genetically linked to said at least one genetic haplotype, wherein said SNP is set forth in Tables 6-8.

12. The method of claim 11, further comprising detecting all SNPs within or genetically linked to said at least one haplotype.

13. The method of claim 1, further comprising genotyping at least one parent of said head of beef cattle for the presence of said at least one genetic haplotype.

14. The method of claim 1, further comprising genotyping both parents of said head of beef cattle.

15. The method of claim 1, wherein said head of beef cattle is a Bos indicus or a Bos taurus head of beef cattle.

16. The method of claim 1, wherein said head of beef cattle is a hybrid of a Bos indicus species and a Bos taurus species.

17. The method of claim 16, wherein said Bos indicus species further comprises a Nellore head of cattle.

18. The method of claim 16, wherein said Bos taurus species further comprises an Angus head of cattle.

19. The method of claim 1, further comprising genotyping a population of beef cattle for the presence of said at least one genetic haplotype.

20. The method of claim 1, further comprising breeding the selected head of beef cattle comprising said at least one genetic haplotype with a second head of beef cattle to obtain a progeny head of beef cattle with increased feed efficiency relative to a head of beef cattle of the same breed lacking said at least one genetic haplotype.

21. The method of claim 20, further comprising breeding the progeny head of beef cattle with increased feed efficiency to a second head of beef cattle to produce a progeny of a further generation comprising said increased feed efficiency.

22. The method of claim 1, wherein the presence of said at least one genetic haplotype conferring said increased feed efficiency is detected by assaying of genetic material from the head of beef cattle.

23. The method of claim 1, wherein said haplotype of (a) comprises the ACTN3 gene.

24. The method of claim 1, wherein said haplotype of (b) comprises the FN1 gene.

25. The method of claim 1, wherein said haplotype of (c) comprises the CAPN2 gene.

26. The method of claim 22, wherein said assaying is carried out by PCR.

27. A method of predicting increased feed efficiency in a head of beef cattle comprising genotyping the head of beef cattle for the presence in the genome of at least one genetic haplotype conferring said increased feed efficiency selected from the group consisting of:
(a) a haplotype located in a region on bovine chromosome 29 defined by positions 45230630 and 45242406;
(b) a haplotype located in a region on bovine chromosome 2 defined by positions 103881402 and 103950562; and
(c) a haplotype located in a region on bovine chromosome 16 defined by positions 27781671 and 27840011.

28. The method of claim 27, wherein said head of beef cattle is a Bos indicus or a Bos taurus head of beef cattle.

29. The method of claim 27, wherein said head of beef cattle is a hybrid between a Bos indicus species and a Bos taurus species.

30. The method of claim 29, wherein said Bos indicus species further comprises a Nellore head of cattle.

31. The method of claim 29, wherein said Bos taurus species further comprises further comprises an Angus head of cattle.

32. A method of determining the breeding value of a head of cattle comprising:
(a) genotyping the head of beef cattle to determine the presence in the genome of at least one genetic haplotype conferring said increased feed efficiency selected from the group consisting of:
(i) a haplotype located in a region on bovine chromosome 29 defined by positions 45230630 and 45242406;
(ii) a haplotype located in a region on bovine chromosome 2 defined by positions 103881402 and 103950562; and
(iii) a haplotype located in a region on bovine chromosome 16 defined by positions 27781671 and 27840011;
(b) determining an estimated breeding value for the individual; and
(c) selecting at least a first individual with a desired estimated breeding value for breeding.

33. The method of claim 32, further comprising:
(d) breeding said individual to a second individual to obtain progeny.

34. A kit for identifying a head of beef cattle with increased feed efficiency, the kit comprising:
(a) at least one primer that amplifies a SNP within or genetically linked to a haplotype conferring said increased feed efficiency selected from the group consisting of:
(i) a haplotype located in a region on bovine chromosome 29 defined by positions 45230630 and 45242406;
(ii) a haplotype located in a region on bovine chromosome 2 defined by positions 103881402 and 103950562; and
(iii) a haplotype located in a region on bovine chromosome 16 defined by positions 27781671 and 27840011;
wherein said SNP is set forth in Tables 6-8; and
(b) a reaction reagent.
